(19) United States
(12) Patent Application Publication
(10) Pub. No.: US 2022/0354840 A1
(43) Pub. Date: Nov. 10, 2022

(54) NICOTINE POUCH

(71) Applicant: Enorama Pharma AB, Malmö (SE)

(72) Inventors: Lucas Lissner, Lund (SE); Yana Falk, Malmö (SE)

(73) Assignee: Enorama Pharma AB, Malmö (SE)

(21) Appl. No.: 17/761,918

(22) PCT Filed: Sep. 17, 2020
(86) PCT No.: PCT/EP2020/075997

§ 371 Date: Mar. 18, 2022

(30) Foreign Application Priority Data
Sep. 18, 2019 (SE) 1951054-4

(51) Int. Cl. A61K 31/465; A61K 9/14; A61K 47/38; A24F 23/02; A61K 47/18; A61K 47/58

(52) U.S. Cl. CPC A61K 31/465; A61K 9/14; A61K 47/38; A24F 23/02; A61K 47/183; A61K 47/585

(57) ABSTRACT

A product for oral delivery of nicotine comprising a powder composition of a nicotine source, at least one pH adjusting agent, and at least one filler, contained in a pouch which is permeable to saliva and to components of the powder composition once dissolved in saliva, wherein the composition has a solubility of less than 5 g/100 ml. The invention also relates to a method for preparing said product and also the use of said product.

### FIELD OF THE INVENTION

The present invention relates to pouches for oral delivery of nicotine.

### TECHNICAL BACKGROUND

Smoking of tobacco is associated with health hazards which are not related to the administration of nicotine per se. While nicotine is highly addictive, the most important risk associated with tobacco use come from the multitude of substances formed and released during the combustion of tobacco, e.g. carcinogens such as nitrosamines and various tar components.

It is generally accepted that the difficulty to quit smoking results from smokers being addicted to the constituent nicotine. It is therefore desirable to use alternative means of delivering nicotine to facilitate reduction of or cessation from smoking. Such products should provide nicotine with no or minimal content of other potential hazardous components associated with tobacco use. A number of alternative nicotine administration forms are known such as gum, patch, nasal or oral spray, lozenge and oral pouch. For these products the purity of the constituent nicotine is important for potential toxicity aspects. Also, the taste of the product is clearly important for consumer acceptance and preference.

During the smoking of a cigarette, nicotine is readily absorbed into the smoker's blood via the large surface of the lungs, and it starts affecting the brain within 10 seconds. The result is increased levels of epinephrine as well as dopamine, a neurotransmitter controlling the brain's pleasure center leading to, among other things, “smoker's satisfaction”. In contrast, products such as nicotine gum, patch, spray, lozenge and pouch release the nicotine in the oral cavity where it may be absorbed quickly through the oral mucosa or it may be partially swallowed leading to a slower and less efficient absorption via the stomach. For these products, the speed as well as the place of nicotine release (oral mucosa vs stomach) are obviously important. Even for a focused, optimized release of nicotine to the oral mucosa, the onset of action will still be in the order of minutes rather than seconds as facilitated by absorption via the lung.

In addition to taste, purity and the onset of action discussed above, the chemical stability of the nicotine molecule is important for its performance in the oral delivery system. In its free base form nicotine is volatile and rapidly undergoes oxidation leading to discoloration and partial loss of activity. To deal with these issues, nicotine base is sometimes absorbed to appropriate carriers and/or non-covalently bound in salts or in complex with ion exchange resins. While this improves the stability of the constituent nicotine, it obviously also affects the rate of nicotine release from the delivery system. Absorption of the nicotine molecule across the oral mucosa requires the neutral form which predominate only at pH values above 8. Consequently, most delivery systems contain a pH controlling substance such as a buffer which brings the pH into the desired range once the nicotine is released from the delivery system. The rate of local pH adjustment in the mouth becomes yet another parameter deciding the overall performance of the nicotine delivery system.

U.S. Pat. No. 9,402,810 describes a pouch based on nicotine extracted from tobacco and subsequently stabilized in the form of a tartrate salt. The pouch described in U.S. Pat. No. 9,907,748 is also based on nicotine extracted from tobacco, in this case stabilized via binding to polacrilex resin. However, for stability reasons, U.S. Pat. No. 9,907,748 teaches that the pH controlling agent is being sorbed onto the inactive cellulose carrier to avoid contact with the nicotine salt in the dry pouch. Further, WO 2018/233795 also uses nicotine extracted from tobacco, and a sugar alcohol sweetener is often used as carrier for the nicotine.

One aim of the present invention is to provide an improved nicotine pouch that at least overcomes some of the disadvantages with the products currently on the market.

### SUMMARY OF THE INVENTION

The stated purpose above is achieved by a product for oral delivery of nicotine comprising a powder composition of:
a nicotine source;
at least one pH adjusting agent; and
at least one filler,
contained in a pouch which is permeable to saliva and to components of the powder composition once dissolved in saliva, wherein the composition has a solubility of less than 5 g/100 ml. In a preferred embodiment the composition has a solubility of less than 2.5 g/100 ml, more preferably less than 1.0 g/100 ml.

The stated purpose above may also be achieved by a product for oral delivery of nicotine comprising a powder composition of:
a nicotine source;
at least one pH adjusting agent; and
at least one filler,
contained in a pouch which is permeable to saliva and to components of the powder composition once dissolved in saliva, wherein the composition has a solubility in water at 25 degrees Celsius and pH 7 of less than 5 g/100 ml. In a preferred embodiment the composition has a solubility of less than 2.5 g/100 ml, more preferably less than 1.0 g/100 ml.

In relation to the above it should be emphasized that none of the pouches described in U.S. Pat. Nos. 9,402,810 or 9,907,748 are providing an optimal combination of nicotine stability, release and uptake profile. U.S. Pat. No. 9,402,810, uses nicotine in the form of a salt. In contrast, nicotine bound to an ion-exchange resin facilitates controlled release and help maximizing the fraction of the dose being absorbed via the mucous membranes in the mouth rather than being swallowed. U.S. Pat. No. 9,907,748 does employ nicotine bound to polacrilex resin. However, in order to minimize contact between nicotine polacrilex (NPR) and buffer in the dry pouch, the buffer is sorbed onto the inactive cellulose carrier. In the presence of saliva, this will prolong the time taken before the buffer makes contact with the nicotine polacrilex. As shown herein, the pouch described in U.S. Pat. No. 9,907,748 releases nicotine relatively slow and more incomplete over a 30-minutes window.

Further, the pouch described in WO 2018/233795A1 uses an excess of polyalcohols which act as the nicotine carrier and also to increase the water solubility of the composition in the pouch. Since it will be difficult to control the water supply in form of saliva for each end-user the high amounts of soluble carrier will possibly lead to loss of nicotine to the stomach due to swallowing rather than absorption through the mucous membrane in the mouth.

In WO 2018/233795A1, a release modifier such as magnesium stearate is needed to slow the release of nicotine from the compositions containing high levels of polyalcohols. In contrast, the proportion of microcrystalline cellulose and (lower level) polyalcohols used herein allows for a controlled nicotine release without the need for specific release modifiers such as magnesium stearate.

In contrast, the proportion of microcrystalline cellulose and much lower level of polyalcohols used herein allows for a controlled nicotine release without the need for specific release modifiers such as magnesium stearate.

In one embodiment the present invention relates to a product wherein the nicotine source has been derived by extraction from tobacco or by chemical synthesis. The nicotine source according to the present invention may be stabilized in the form of a nicotine salt or bound to an ion exchange resin.

In one embodiment the present invention the ion exchange resin may be a weak cation exchange resin, such as polacrilex.

In one embodiment of the present invention the nicotine source may be nicotine polacrilex.

In another embodiment of the present invention the nicotine source may be nicotine benzoate or nicotine maleate.

In a further embodiment the present invention relates to a product wherein the at least one pH adjusting agent is a buffer comprising carbonates, bicarbonates, borates, glycinates, ammonium, phosphates, hydroxides, tris, or mixtures thereof. The pH adjusting agent of the present invention may be a buffer comprising sodium carbonate, sodium bicarbonate, potassium carbonate, or mixtures thereof.

In yet another embodiment the present invention relates to a product wherein the at least one filler comprises polyalcohols, sweeteners, polysaccharides, cellulose, microcrystalline cellulose, natural fibers, flavors, or mixtures thereof. In one embodiment of the present invention the at least one filler may comprise a microcrystalline cellulose, such as Avicel PH-200.

In another embodiment the at least one filler may comprise a mixture of microcrystalline cellulose 90M(102) and the polysaccharide sodium alginate.

The sweeteners of the present invention may be selected from the group consisting of mannitol, xylitol, maltitol, sucralose, acesulfam potassium, aspartame, steviol glycosides, or mixtures thereof. Further, the flavors of the present invention may be selected from the group consisting of menthol, cinnamon, mint, peppermint, spearmint, apple, cherry, melon, mango, peach, passion fruit, orange, blood orange, grape fruit, mandarin, tangerine, licorice, ginger, vanilla, wintergreen, lemon, lime, strawberry, raspberry, salmonberry, lingonberry, cranberry, blueberry, muscadine berry, sea buckthorn, chocolate, coffee, mocha, or mixtures thereof.

In some embodiments, the pouch material may comprise additionally added moisture components in the form of water, propylene glycol, glycerin, or mixtures thereof. In the present invention all reported weight proportions of powder components are based on the total weight (including added moisture components if any).

In one embodiment the present invention relates to a product wherein the weight proportions of the powder composition are; 0.5-2 wt % of the nicotine source calculated as nicotine base, 4-15 wt % of the pH adjusting agent and 70-85 wt % of the at least one filler. The product of the present invention may comprise microcrystalline cellulose in the amount of 40-80 wt %, preferably 43-75 wt %. Further, the product of the present invention may comprise nicotine in an amount ranging from 0.5-2 wt % calculated as nicotine base. Furthermore, the product of the present invention may comprise at least one pH adjusting agent in an amount 4-15 wt %, preferably 5-13 wt %, more preferably 5-11 wt %. The product of the present invention may comprise at least one polyalcohol in an amount of 5-50 wt %, preferably 6-45 wt %, more preferably 8-40 wt %. The weight proportions of the nicotine source in relation to the amount of the pH adjusting agent and fillers, especially the microcrystalline cellulose, is of great importance in order to achieve the optimum nicotine release from the pouch.

In another embodiment the present invention relates to a product wherein the weight proportions of the powder composition are; 0.15-2 wt % of the nicotine source calculated as nicotine base, 2-15 wt % of the pH adjusting agent and 40-85 wt % of the at least one filler. The product of the present invention may comprise microcrystalline cellulose in the amount of 40-80 wt %, preferably 30-70 wt %. Further, the product of the present invention may comprise nicotine in an amount ranging from 0.15-2 wt % calculated as nicotine base. Furthermore, the product of the present invention may comprise at least one pH adjusting agent in an amount 1-15 wt %, preferably 2-11 wt %. The product of the present invention may optionally comprise at least one polyalcohol in an amount of 0-50 wt %, preferably 0-40 wt %, moisturizing agents such as propylene glycol and/or glycerin in the amount of 0-15wt % and water in the amount of 0-35%.

In one preferred embodiment the present invention relates to a product wherein the at least one filler comprises microcrystalline cellulose, such as Avicel PH-200 or Hicel 90M (102).

In one preferred embodiment the present invention relates to a product wherein the weight proportions of the powder composition are; 0.15-2 wt % of the nicotine source calculated as nicotine base, 2-15 wt % of the pH adjusting agent and 40-85 wt % of the at least one filler.

In one preferred embodiment the present invention relates to a product wherein the product comprises microcrystalline cellulose in the amount of 30-80 wt %.

In one preferred embodiment the present invention relates to a product wherein the pouch has a nicotine release fraction of 50-100% after 30 min.

Further the invention also relates to a product wherein the pouch is made of a non-woven material. In another embodiment the pouch is made from a biodegradable material. In a preferred embodiment the pouch is made from a home compostable material.

Another aspect of the present invention relates to a method for preparing the product according to the present invention, wherein it comprises the steps of:

a) providing the nicotine source, the at least one pH adjusting agent and the at least one filler, in powder form;

b) mixing the powders provided in step a);

c) optionally sieving the powder mixture of step b) into a powder composition; and

d) optionally filling the powder composition into a pouch.

Another aspect of the present invention relates to a method for preparing the product according to the present invention, wherein it comprises the steps of:

a) providing the nicotine source, the at least one pH adjusting agent and the at least one filler, in powder form;

b) mixing the powders provided in step a);

c) optionally sieving the powder mixture of step b) into a powder composition; and

d) optionally filling the powder composition into a pouch.

e) optionally addition of the moisture components to step b) in the form of water, propylene glycol, glycerin, or mixtures thereof.

In a further aspect the present invention relates the use of the product according to the present invention for oral delivery of nicotine.

### SHORT DESCRIPTION OF THE DRAWINGS

In the figures below, the nicotine release from products according to the disclosed invention as well as reference products are presented.

FIG. 1 shows nicotine release profiles for the pouch products according to the present invention: Example 1 (Batch A, filled squares); Reference 1A (2 mg Nicotine, left triangles) and Reference 1B (4 mg Nicotine, right triangles); Reference 2A (3 mg Nicotine, up triangles) and Reference 2B (6 mg nicotine, down triangles).

FIG. 2 shows nicotine release data for the pouch product according to the present invention, Example 1 (Batch B, filled circles with error bars) and stability data: 1 month, T=21-22° C., 60-62% RH (black empty circle with corresponding error bars).

FIG. 3 shows nicotine release data for the analyzed pouch products from different batches (A, B and C) according to the present invention, Example 1. Pouch products with different concentration range for the buffer agents (Example 2, black star with error bars), nicotine polacrilex (Example 3, black cross with error bars) and fillers (Example 4, black pentagram with error bars) are analyzed and compared for the nicotine release at 30 min.

FIG. 3 shows nicotine release data for the analyzed pouch products with different fillers and moisturizing agents (Example 5, open circle with error bars) are analyzed and compared for the nicotine release at 30 min.

FIGS. 4A-C show the results from the degradation study of the pouch of the present invention, the chromatograms from the HPLC-UV analysis of the pouch sample stored at 21-22° C./60-62% RH for up to 3 months (FIG. 4A), sample with the same excipients but without nicotine API (FIG. 4B) and sample of nicotine polacrilex 20% API (FIG. 4C).

FIGS. 5A-B show the results from the degradation study of the pouch of the present invention, the chromatograms from the HPLC-UV analysis of a pre-stressed sample of nicotine (FIG. 5A) was compared to the chromatogram of nicotine bitartrate dihydrate spiked into the reference sample without API (FIG. 5B).

### DETAILED DESCRIPTION OF THE INVENTION

Although individual features may be included in different embodiments, these may possibly be combined in other ways, and the inclusion in different embodiments does not imply that a combination of features is not feasible. In addition, singular references do not exclude a plurality. In the context of the present invention, the terms “a”, and “an” do not preclude a plurality.

The design of a nicotine delivering pouch require co-optimization of several properties, in particular nicotine stability and release as well as taste masking. Surprisingly, it has been found that a pouch based on nicotine polacrilex according to the present invention is both stable and can release nicotine as fast as a pouch based on a typical salt such as nicotine tartrate. Polacrilex is an insoluble, weakly acidic cation exchange resin. The matrix contains methacrylic acid capable of exchanging hydrogen for nicotine cations, e.g. Amberlite IRP64® or Doshion P-551®.

The pouch according to the present invention contains a powdered mix of a nicotine source, at least one pH controlling agent and at least one filler, such as microcrystalline cellulose, as well as sweeteners and flavors.

In a preferred embodiment according to the present invention, the buffer powder is directly mixed with the other powder components of the pouch to facilitate rapid pH adjustment upon contact with the saliva.

Nicotine may be derived either by extraction from tobacco or from chemical synthesis. In comparison with the synthetic nicotine, the extracted material contains small amounts of tobacco-derived non-nicotine alkaloids. While these impurities may be toxic, they likely contribute difficulties for the taste masking of the product. Although both sources of nicotine are acceptable embodiments of this invention, the synthetic material provides additional advantages in terms of taste setting. Hence, in one preferred embodiment the nicotine is derived from chemical synthesis. In an advantageous embodiment of the present invention the purity of nicotine is in the range of 99.00-99.99%, preferably in the range of 99.60-99.99%.

According to one embodiment of the present nicotine may be in the form of nicotine polacrilex or a nicotine salt, such benzoate or maleate. In another embodiment nicotine polacrilex is preferred due to the favorable combination of nicotine stability and fast release affected by the direct mix with the pH controlling agent.

Further, the pH controlling agent according to the present invention may be a carbonate buffer, such as sodium carbonate, sodium bicarbonate, potassium carbonate, or mixtures thereof. In other preferred embodiments the buffer may be a borate, such as sodium tetraborate; phosphate, such as mono-, di- or trihydrogenphosphate; or tris(hydroxymethyl)aminomethane (tris). Preferred buffer agents according to the present invention are mixtures of sodium carbonate and sodium bicarbonate.

In addition to the nicotine source and pH controlling agents, the pouch of the present invention also contains at least one filler.

Fillers according to the present invention may include polyalcohols, sweeteners, polysaccharides, cellulose, microcrystalline cellulose, natural fibers, flavors. Polyalcohols, or sugar alcohols are a class of polyols that are commonly obtained by the hydrogenation of sugars. Further, the polyalcohols comprise a lower caloric content than sugars and are also, in general, less sweet. Polysaccharides are polymeric carbohydrate molecules composed of long chains of monosaccharide units bound together by glycosidic linkages, and on hydrolysis give the constituent monosaccharides or oligosaccharides. They range in structure from linear to highly branched. Examples include storage polysaccharides such as starch and glycogen, and structural polysaccharides such as cellulose, chitin and alginate. Cellulose is a very common natural polymer and it is the largest component of plant cell walls. The cellulose usually consists of polymer chains that are 10,000-15,000 molecules long. Microcrystalline cellulose (MCC) is synthetically made and consists of chains that are between 100 and 300 molecules long. With natural fibers means fibers that are produced by plants, animals, or geological processes.

The at least one filler of the present invention may be microcrystalline cellulose, such as Avicel PH-200, which may have solubility of less than 5 g/100 ml. Fillers with poor water solubility limit the water solubility of the powder composition in the pouch and, therefore, do not allow the pouch content to be completely dissolved when in contact with water supplied in the form of saliva. Thus, the use of fillers with poor water solubility, such as Avicel PH-200, may result in a relatively slow release and direct absorption of nicotine through the mucus membrane in the mouth. In a preferred embodiment the water solubility of microcrystalline cellulose is less than 2.5 g/100 ml, more preferable less than 1.0 g/100 ml, even more preferable less than 0.75 g/100 ml, most preferable less than 0.58 g/100 ml measured in H₂O at 25° C. and pH 7. This contributes greatly to the advantageous nicotine release profile of the pouch according to the present invention.

The at least one filler in one embodiment of the present invention may be microcrystalline cellulose, such as Avicel PH-200. In another embodiment of the present invention, the at least one filler may comprise a mixture of microcrystalline cellulose 90M(102) and the polysaccharide sodium alginate. Both embodiments of the present invention may have a water solubility of less than 5 g/100 ml. Fillers with poor water solubility limit the water solubility of the powder composition in the pouch and, therefore, do not allow the pouch content to be completely dissolved when in contact with water supplied in the form of saliva. Thus, the use of fillers with poor water solubility, such as Avicel PH-200 or Hicel 90M(102), may result in a relativley slow release and direct absorption of nicotine through the mucus membrane in the mouth. In a preferred embodiment the water solubility of microcrystalline cellulose is less than 2.5 g/100 ml, more preferable less than 1.0 g/100 ml measured in H₂O at 25° C. This contributes greatly to the advantageous nicotine release profile of the pouch according to the present invention.

Sweeteners according to the present invention may comprise mannitol, xylitol, maltitol, sucralose, acesulfam potassium, aspartame, steviol glycosides, or mixtures thereof. Flavors according to the present invention may include menthol, cinnamon, mint, peppermint, spearmint, apple, cherry, melon, mango, peach, passion fruit, orange, blood orange, grape fruit, mandarin, tangerine, licorice, ginger, vanilla, wintergreen, lemon, lime, strawberry, raspberry, salmonberry, lingonberry, cranberry, blueberry, muscadine berry, sea buckthorn, chocolate, coffee, mocha, or mixtures thereof.

In an advantageous embodiment of the present invention, the distinct balance of microcrystalline cellulose and polyalcohol levels contribute to the favorable release profile of nicotine from the matrix.

### EXAMPLES

The formulation of the present invention is exemplary and should be considered as an inspiration to obtain a similar outcome within the scope of the present invention. A specification of suppliers for the different ingredients used in the present application is disclosed in Table 1.

A specification of suppliers for the different ingredients used in the present application is disclosed in Table 1B.

TABLE 1
Specification of suppliers for the different ingredients.

| Ingredients | Supplier | Form | Particle size | Solubility in water | Function |
|---|---|---|---|---|---|
| Nicotine Polacrilex | Siegfried | Solid/white | 96%: <710 μm | Insoluble | Nicotine source |
| (NPR) 20% | | fine powder | 90%: <210 μm | | |
| Xylitol | Sigma-Aldrich | Solid/white | — | Very soluble | Filler Sweetener |
| | | crystalline | | 642 mg/ml at 25° C. | |
| | | powder | | | |
| D-Mannitol | Sigma-Aldrich | Solid/white | — | Very soluble | Filler Sweetener |
| | | crystalline | | 216 mg/ml at 25° C. | |
| | | powder | | | |
| Avicel PH-200 | FMC | Solid/white | 200 μm | Insoluble | Filler |
| Microcrystalline | | free-flowing | | (water soluble | |
| cellulose | | powder | | substances: | |
| | | | | NMT 12.5 mg/5 g | |
| | | | | (0.25%) at 25° C.) | |
| Sodium Carbonate | Sigma-Aldrich | Solid/white | — | Very soluble | Buffering agent |
| Monohydrate | | powder | | 307 mg/ml at 25° C. | |
| Sodium Bicarbonate | Sigma-Aldrich | Solid/white | — | 103 mg/ml at 25° C. | Buffering agent |
| | | powder | | | |
| L-Menthol | Sigma-Aldrich | Solid/transparent | — | Low soluble | Flavoring agent |
| | | large crystals | | 0.49 mg/ml at 25° C. | |
| Apple flavor | Firmenich | Solid/colored | — | — | Flavoring agent |
| | | powder | | | |
| Cinnamon flavor | Firmenich | Solid/colored | — | — | Flavoring agent |
| | | powder | | | |
| Acesulfame K | Celanese | Solid/white | Min 95%: <1000 μm | High solubility: | Sweetener |
| | | crystalline | | 270 mg/ml at 20° C. | |
| | | powder | | | |

TABLE 1B
Specification of suppliers for the different ingredients.

| Ingredients | Supplier | Form | Particle size | Solubility in water | Function |
|---|---|---|---|---|---|
| Nicotine Polacrilex | Siegfried | Solid/white | 96%: <710 μm | Insoluble | Nicotine source |
| (NPR) 20% | | fine powder | 90%: <210 μm | | |
| Xylitol | Sigma-Aldrich | Solid/white | — | Very soluble | Filler Sweetener |
| | | crystalline | | 642 mg/ml at 25° C. | |
| | | powder | | | |
| D-Mannitol | Sigma-Aldrich | Solid/white | — | Very soluble | Filler Sweetener |
| | | crystalline | | 216 mg/ml at 25° C. | |
| | | powder | | | |
| Avicel PH-200 | FMC | Solid/white | 200 μm | Insoluble | Filler |
| Microcrystalline | | free-flowing | | (water soluble | |
| cellulose | | powder | | substances: | |
| | | | | NMT 12.5 mg/5 g | |
| | | | | (0.25%) at 25° C.) | |
| Hicel 90M (102) | Brenntag | Solid/white | 90 μm | Insoluble | Filler |
| Microcrystalline | | free flowing | | (water soluble | |
| cellulose | | powder | | substances: <0.24%) | |
| Satialgine S 900NS | Brenntag | Solid/creamy- | — | Soluble | Filler |
| Sodium Alginate | | white to light- | | | |
| | | brown powder | | | |
| Sodium Carbonate | Brenntag | Solid/white | — | Very soluble | Buffering agent |
| Monohydrate | | powder | | 307 mg/ml at 25° C. | |
| Sodium Bicarbonate | Sigma-Aldrich | Solid/white | — | 103 mg/ml at 25° C. | Buffering agent |
| | | powder | | | |
| L-Menthol | Sigma-Aldrich | Solid/transparent | — | Low soluble | Flavoring agent |
| | | large crystals | | 0.49 mg/ml at 25° C. | |
| Menthol flavor | Firmenich | Solid powder | — | — | Flavoring agent |
| Flavors | Firmenich | Solid/white | — | — | Flavoring agent |
| | | or colored | | | |
| | | powder | | | |
| Acesulfame K | Celanese | Solid/white | Min 95%: <1000 μm | High solubility: | Sweetener |
| | | crystalline | | 270 mg/ml at 20° C. | |
| | | powder | | | |
| Ammonium Chloride | Brenntag | White | — | — | Taste enhancer |
| | | crystalline | | | |
| | | powder | | | |
| Propylene glycol | Caldic | Clear colorless | — | Soluble | Moisturizing agent |
| | | odorless liquid | | | |

Product Manufacturing

A product with a composition according to the present invention is manufactured in the following manner: all ingredients, that are in powder form, are mixed and sieved into the final powder mixture. The resulting powder is filled into a pouch made of a non-woven material or cloth. Non-woven material, cloths or fabrics may be defined as sheet or web structures bonded together by entangling fibers or filaments, and by perforating films, mechanically, thermally, or chemically. They are flat, porous sheets that are made directly from separate fibers or from molten plastic or plastic film. They are not made by weaving or knitting and do not require converting the fibers to yarn. The whole manufacture process is performed at ambient conditions of temperature and relative humidity (RH), ranging between 21-25° C. and 30-60% RH, respectively.

Solubility in Water

The water used for solubility measurements is either distilled or filtered via a Millipore Milli-Q system. An unused pouch with weight P0 (g) is placed in a test tube containing 20 ml of water and left on the shaker at room temperature for 1 h. The pouch is isolated and dried to constant weight Pe (g) in air at room temperature. Three replicates for each batch are used in the solubility measurements. The water solubility of the pouch composition is then (P0-Pe) (g) per 20 ml water.

In-Vitro Release Experiments

An experimental method for the determination of the nicotine release from pouch was performed using UV spectrophotometric methodology. Pouch sample preparation and method of analysis were adapted and modified from the published methods for pouch analysis (see U.S. Pat. No. 9,402,810).

Briefly, a pouch sample is placed on the middle of the filter paper which is pre-soaked in 20 ml water phase in a Petri dish. The whole setup is covered by lid to maintain the relative humidity level and prevent water evaporation. As the pouch starts to absorb the water, the nicotine polacrilex is dissolved by liquid and the released nicotine diffuses into the external water phase.

This is conducted at specific incubation times, i.e. 10, 20 and 30 min (note a new pouch for each time). Three replicates from each batch of Example 1 are used in the experiment for each time. After each analyzed timepoint, the pouch sample is placed into the test tube containing 15 ml of water and left on the shaker for 1.5-2 hours.

A number of unused pouches from the same batch are also placed into the test tube, containing 15 ml purified water, and left on the shaker for 1.5-2 hours for analysis of the total nicotine content.

Afterwards, the water phase containing nicotine is analyzed for the residual content of nicotine using the UV spectrophotometer (Shimadzu, UV1800). First, each sampling of the water phase from the test tube is filtered using the 0.45 μm filter unit. The filtered solution is then diluted to the suitable concentration, and the sample absorbance is measured. The measurements are conducted at photometric mode with fixed wavelength of 260 nm.

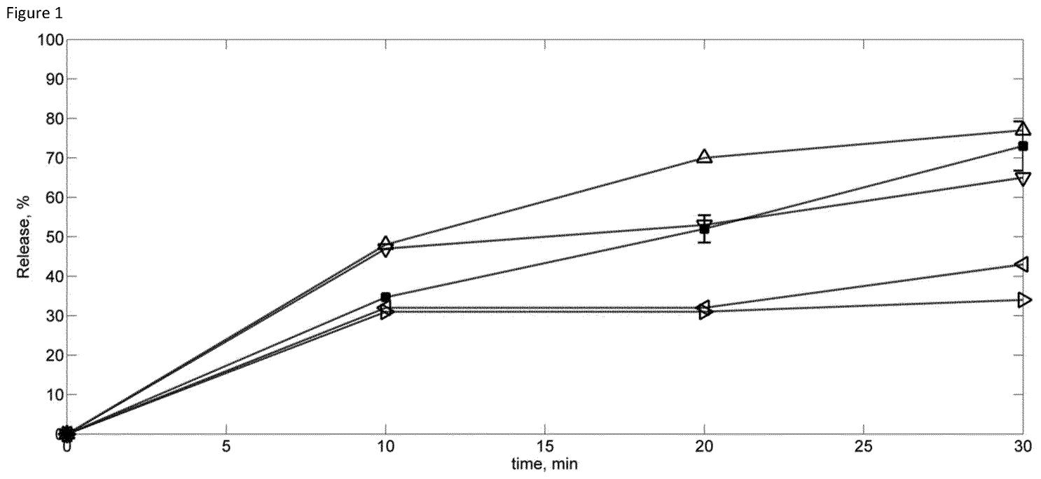

The nicotine concentration for the analyzed samples (non-used and used pouches) is calculated from a calibration curve, and the total and residual nicotine content is determined. The release of nicotine at specific time is calculated by subtracting the ‘residual nicotine content’ from the ‘total nicotine content’ and dividing it by the ‘total nicotine content’. Table 2 shows four different example compositions (Examples 1-4) according to the present invention that have been analyzed. Table 3 summarizes the obtained data for the nicotine release for the four example compositions of the present invention and reference pouch samples. The obtained data are plotted and shown in FIGS. 1-3.

Table 2B shows five different example compositions (Examples 1-5) according to the present invention that have been analyzed. Table 3B summarizes the obtained data for the nicotine release for the five example compositions of the present invention and reference pouch samples. The obtained data are plotted and shown in FIGS. 1-3.

TABLE 2
Formulation compositions of the present invention. The pouch
weight is 0.4 g. The nicotine dose corresponds to the 4 mg per
pouch (Examples 1, 2 and 4) and 8 mg per pouch (Example 3).

| | Example | Example | Example | Example |
|---|---|---|---|---|
| Content | 1 | 2 | 3 | 4 |
| NPR 20%* | 5 | 5 | 10 | 5 |
| Xylitol | 16 | 16 | 16 | 4 |
| Mannitol | 16 | 16 | 16 | 4 |
| Avicel | 48 | 53.2 | 43 | 72 |
| PH-200 | | | | |
| Sodium | 4.0 | 2.0 | 4.0 | 4.0 |
| Carbonate | | | | |
| Sodium | 6.4 | 3.2 | 6.4 | 6.4 |
| Bicarbonate | | | | |
| Menthol | 0.2 | 0.2 | 0.2 | 0.2 |
| Apple flavor | 2.0 | 2.0 | 2.0 | 2.0 |
| Cinnamon | 2.14 | 2.14 | 2.14 | 2.14 |
| flavor | | | | |
| Ace K** | 0.26 | 0.26 | 0.26 | 0.26 |

*NPR—Nicotine polacrilex;
**Ace K—Acesulfame potassium.

TABLE 2B
Formulation composition of the present invention. The pouch
weight is 0.4 g (Examples 1-4) and 0.6 g (Example 5). The
nicotine dose corresponds to the 4 mg per pouch (Examples
1, 2, 4 and 5) and 8 mg per pouch (Example 3).

| | Example | Example | Example | Example | Example |
|---|---|---|---|---|---|
| Content | 1 | 2 | 3 | 4 | 5 |
| NPR 20%* | 5 | 5 | 10 | 5 | 3.3 |
| Xylitol | 16 | 16 | 16 | 4 | 0 |
| Mannitol | 16 | 16 | 16 | 4 | 0 |
| Sodium | 0 | 0 | 0 | 0 | 5 |
| Alginate | | | | | |
| Avicel | 48 | 53.2 | 43 | 72 | 0 |
| PH-200 | | | | | |
| Hicel 90M | 0 | 0 | 0 | 0 | 48.9 |
| (102) | | | | | |
| Sodium | 4.0 | 2.0 | 4.0 | 4.0 | 2.5 |
| Carbonate | | | | | |
| Sodium | 6.4 | 3.2 | 6.4 | 6.4 | 0 |
| Bicarbonate | | | | | |
| Water | 0 | 0 | 0 | 0 | 20.0 |
| Propylene | 0 | 0 | 0 | 0 | 14.5 |
| glycol | | | | | |
| L-Menthol | 0.2 | 0.2 | 0.2 | 0.2 | 0 |
| Menthol | 0 | 0 | 0 | 0 | 0.7 |
| flavor | | | | | |
| Apple flavor | 2.0 | 2.0 | 2.0 | 2.0 | 0 |
| Cinnamon | 2.14 | 2.14 | 2.14 | 2.14 | 0 |
| flavor | | | | | |
| Peppermint | 0 | 0 | 0 | 0 | 3.0 |
| flavor | | | | | |
| Spearmint | 0 | 0 | 0 | 0 | 1.0 |
| flavor | | | | | |
| Ace K** | 0.26 | 0.26 | 0.26 | 0.26 | 0.6 |
| Ammoinium | 0 | 0 | 0 | 0 | 0.5 |
| chloride | | | | | |

*NPR—Nicotine polacrilex;
**Ace K—Acesulfame potassium.

TABLE 3
Pouches according to the present invention together with reference pouches
and their respective measured nicotine release at specific timepoints.

| | Nicotine | Nicotine | Release fraction (%) | | |
|---|---|---|---|---|---|
| | source/ | dose/pouch | X_{released(time)} | | |
| Sample ID | Conc % | mg | 10 min | 20 min | 30 min |

In-Vitro

| | | | | | |
|---|---|---|---|---|---|
| Example 1 Batch A | NPR/1.0% | 4 | 35 | 54 | 75 |
| | | | 34 | 54 | 66 |
| | | | 35 | 48 | 78 |
| Mean value ± Std | | | 35 ± 0.6 | 52 ± 3.5 | 73 ± 6.2 |
| Example 1 Batch B | NPR/1.0% | 4 | 38 | 63 | 61 |
| | | | 51 | 55 | 76 |
| | | | 39 | 56 | 69 |
| Mean value ± Std | | | 43 ± 7.2 | 58 ± 4.3 | 69 ± 7.5 |
| Stability study: | | | — | — | 61 |
| 1 month | | | | | 62 |
| (T = 21 − 22° C., | | | | | 77 |
| 60-62% RH), Batch B | | | | | |
| Mean value ± Std | | | — | — | 67 ± 8.9 |
| Example 1 Batch C | NPR/1.0% | 4 | 22 | 60 | 71 |
| | | | 32 | 49 | 72 |
| | | | 32 | 54 | 86 |
| Mean value ± Std | | | 29 ± 5.8 | 54 ± 5.5 | 76 ± 8.4 |
| Example 2 | NPR/1.0% | 4 | — | — | 69 |
| | | | | | 84 |
| | | | | | 84 |
| Mean value ± Std | | | — | — | 79 ± 8.7 |
| Example 3 | NPR/2.0% | 8 | — | — | 53 |
| | | | | | 49 |
| | | | | | 61 |
| Mean value ± Std | | | — | — | 54 ± 6.1 |
| Example 4 | NPR/1.0% | 4 | — | — | 68 |
| | | | | | 61 |
| | | | | | 60 |
| Mean value ± Std | | | — | — | 63 ± 4.3 |
| Reference 1A | NPR/1.1% | 2 | 32 | 32 | 43 |
| (Zonnic) | | | | | |
| Reference 1B | NPR/2.2% | 4 | 31 | 31 | 34 |
| (Zonnic) | | | | | |
| Reference 2A | Nicotine | 3 | 48 | 70 | 77 |
| (ZYN mini dry) | Salt/0.75% | | | | |
| Reference 2B | Nicotine | 6 | 47 | 53 | 65 |
| (ZYN mini dry) | Salt/1.5% | | | | |

TABLE 3B
Pouches according to the present invention together with reference pouches
and their respective measured nicotine release at specific timepoints.

| | Nicotine | Nicotine | Release fraction (%) | | |
|---|---|---|---|---|---|
| | source/ | dose/pouch | X_{released(time)} | | |
| Sample ID | Conc % | mg | 10 min | 20 min | 30 min |

In-Vitro

| | | | | | |
|---|---|---|---|---|---|
| Example 1 Batch A | NPR/1.0% | 4 | 35 | 54 | 75 |
| | | | 34 | 54 | 66 |
| | | | 35 | 48 | 78 |
| Mean value ± Std | | | 35 ± 0.6 | 52 ± 3.5 | 73 ± 6.2 |
| Example 1 Batch B | NPR/1.0% | 4 | 38 | 63 | 61 |
| | | | 51 | 55 | 76 |
| | | | 39 | 56 | 69 |
| Mean value ± Std | | | 43 ± 7.2 | 58 ± 4.3 | 69 ± 7.5 |
| Stability study: | | | — | — | 61 |
| 1 month | | | | | 62 |
| (T = 21 − 22° C., | | | | | 77 |
| 60-62 % RH), Batch B | | | | | |
| Mean value ± Std | | | — | — | 67 ± 8.9 |
| Example 1 Batch C | NPR/1.0% | 4 | 22 | 60 | 71 |
| | | | 32 | 49 | 72 |
| | | | 32 | 54 | 86 |
| Mean value ± Std | | | 29 ± 5.8 | 54 ± 5.5 | 76 ± 8.4 |
| Example 2 | NPR/1.0% | 4 | — | — | 69 |
| | | | | | 84 |
| | | | | | 84 |
| Mean value ± Std | | | — | — | 79 ± 8.7 |
| Example 3 | NPR/2.0% | 8 | — | — | 53 |
| | | | | | 49 |
| | | | | | 61 |
| Mean value ± Std | | | — | — | 54 ± 6.1 |
| Example 4 | NPR/1.0% | 4 | — | — | 68 |
| | | | | | 61 |
| | | | | | 60 |
| Mean value ± Std | | | — | — | 63 ± 4.3 |
| Example 5 | NPR/0.66% | 4 | — | — | 67 |
| | | | | | 68 |
| | | | | | 70 |
| Mean value ± Std | | | — | — | 68 ± 1.5 |
| Reference 1A | NPR/1.1% | 2 | 32 | 32 | 43 |
| (Zonnic) | | | | | |
| Reference 1B | NPR/2.2% | 4 | 31 | 31 | 34 |
| (Zonnic) | | | | | |
| Reference 2A | Nicotine | 3 | 48 | 70 | 77 |
| (ZYN mini dry) | Salt/0.75% | | | | |
| Reference 2B | Nicotine | 6 | 47 | 53 | 65 |
| (ZYN mini dry) | Salt/1.5% | | | | |

1) Pouch Analysis: a Comparison with Commercially Available Reference Pouches.

FIG. 1 shows that the pouch products of the present invention—Example 1 (filled squares with error bars) results in similar nicotine release at 10 min and higher release of nicotine at 20 min and 30 min as compared to the nicotine release for Reference 1 pouch products containing nicotine polacrilex (right and left triangles). Moreover, all the examples of the pouch products according to the present invention show similar release of nicotine as compared to Reference 2 pouch product, containing nicotine salt (up and down triangles) at 20 min and 30 min.

Pouch based on nicotine polacrilex according to the present invention can release nicotine as fast as a pouch based on a typical salt such as nicotine tartrate. Moreover, pouch based on nicotine polacrilex according to the present invention can release higher amount of nicotine in comparison to other nicotine polacrilex based pouch products.

### 2) Stability Study:

nicotine release at 30 min
degradation product
   Pouch samples from Example 1 were stored in a plastic container with closed (but not hermetically sealed) lid at 21-22° C./60-62% RH for up to 3 months. The release of nicotine at 30 min for the pouch product stored for 1 month at 21-22° C./60-62% RH was analyzed, using the UV spectrophotometrical method described above.

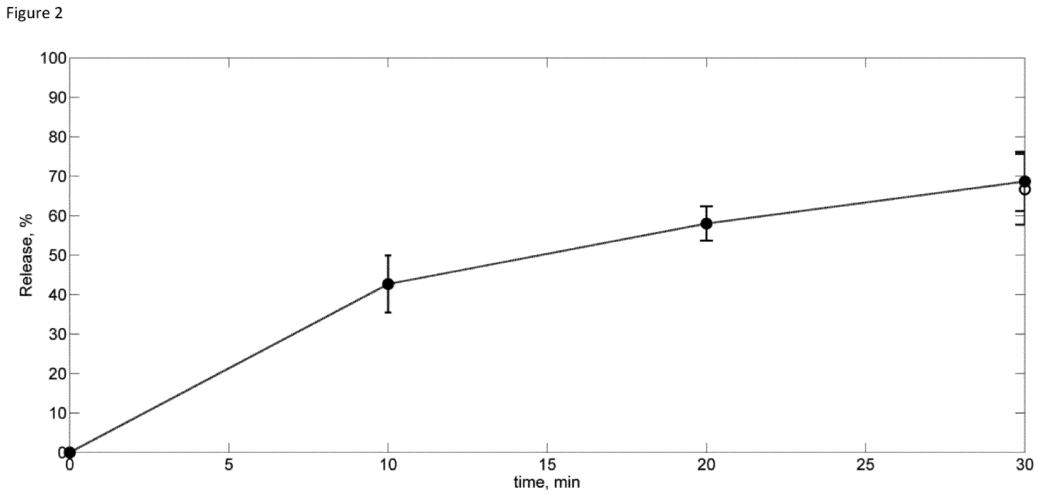

FIG. 2 shows the obtained results where no changes in the nicotine release are observed for the pouch product samples (Example 1) stored for one month at 21-22° C./60-62% RH (black empty circle).

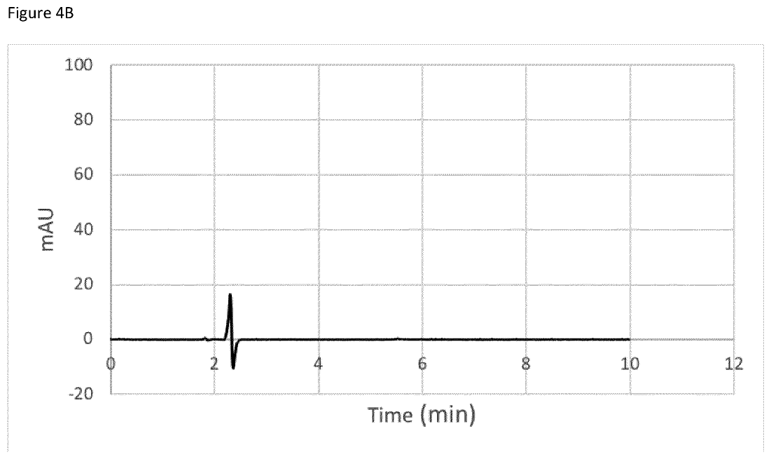

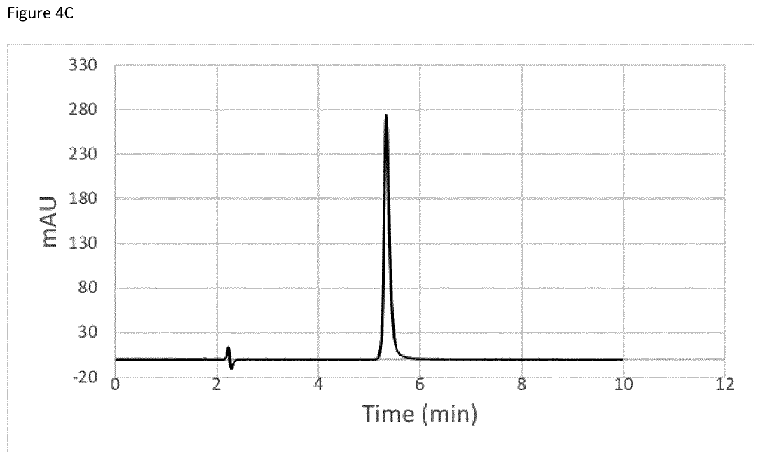

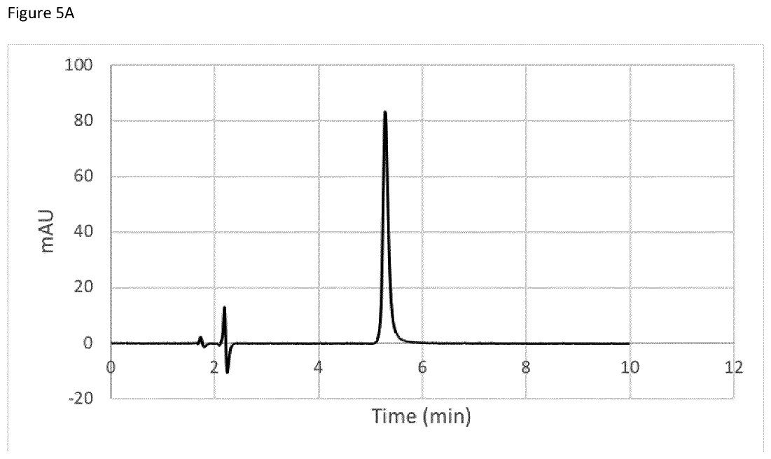

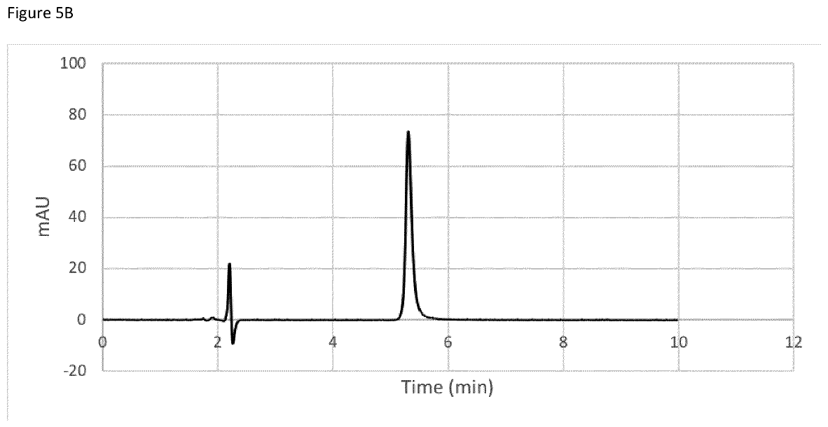

Pouch samples from Example 1 were also analyzed for the detection of potential degradation products, using HPLC-UV method (Agilent 1200 series HPLC). Samples with the same excipients but without the nicotine API and samples with the nicotine API was used to identify reference (blank) peaks. A pre-stressed sample of nicotine was prepared (using elevated temperature and light) to reveal any peaks originating from heat- and light-induced degradation of nicotine. FIGS. 4A-C show the chromatograms from the HPLC-UV analysis of the pouch sample of the present invention stored at 21-22° C./60-62% RH for up to 3 months (FIG. 4A), a pouch sample with the same excipients but without nicotine API (FIG. 4B) and a sample of nicotine polacrilex 20% API (FIG. 4C). The obtained chromatograms show no peaks for the potential degradation products in analyzed samples. The obtained peaks in the chromatogram corresponds to nicotine or solvent injection peak. The chromatogram from the HPLC-UV analysis of pre-stressed sample of nicotine (FIG. 5A), which was compared to the chromatogram of nicotine bitartrate dihydrate spiked into the reference sample without API (FIG. 5B). No additional peaks from potential degradation products were observed in the chromatogram of the pre-stressed sample (FIG. 5A).

Pouch based on nicotine polacrilex according to the present invention is stable and release the same amount of nicotine after 1 month storage as a freshly manufactured pouch based on nicotine polacrilex according to the present invention. Pouch based on nicotine polacrilex according to the present invention is stable and show no potential degradation products in analyzed pouch samples from example 1, stored at 21-22° C./60-62% RH for up to 3 months.

### 3) Concentration Range for the Components:

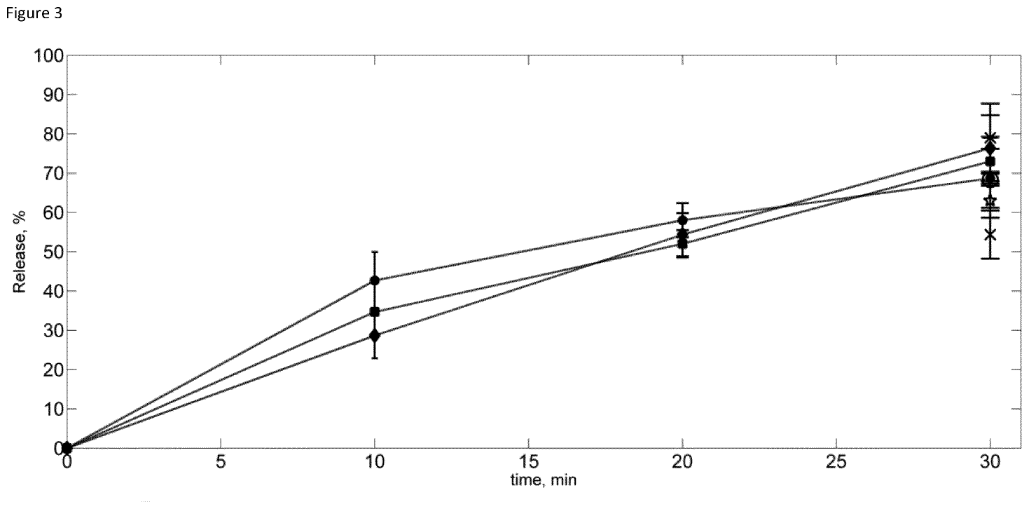

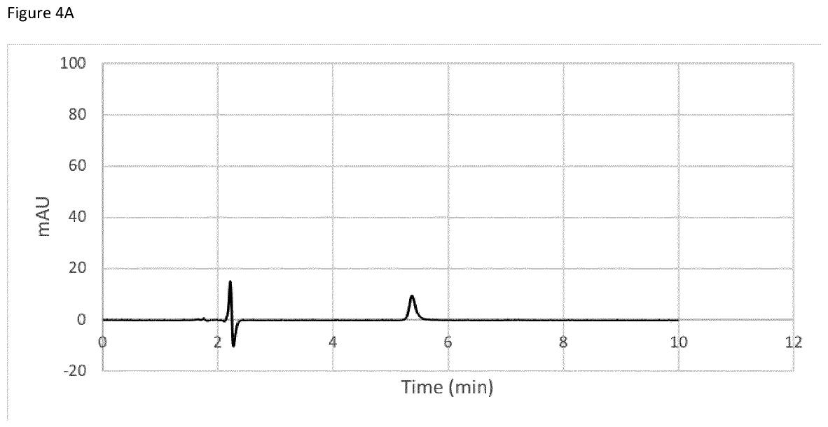

Buffer (reduced twice)
Nicotine polacrilex (increased twice)
Filler (polyalcohols reduced 4 times)
Filler (no polyalcohols, addition of polysaccharide and/or moisturizing agents)
   FIG. 3 shows similar nicotine release at 30 min even if the content of buffer agents is reduced twice in the current pouch formulation recipe (black star with error bars, Example 2).

If the nicotine content is increased twice in the current pouch formulation recipe (Table 2, Example 3), the release of nicotine at 30 min is lower as compared to the release of nicotine of Examples 1, 2 and 4 at 30 min (Table 2). However, similar trend could be observed in the analyzed reference pouch products with higher amount of nicotine (FIG. 1, black triangles). For example, analysis of Reference 1B pouch containing 4 mg NPR (FIG. 1, right triangles) results in lower nicotine release at 30 min as compared to the Reference 1A pouch containing 2 mg NPR (FIG. 1, left triangles). Similar scenario is observed for Reference 2 pouch products: pouch, containing 6 mg nicotine salt (FIG. 1, down triangles) shows lower release of nicotine at 30 min than pouch containing 3 mg nicotine salt (FIG. 1, up triangles).

FIG. 3 shows slightly lower release of nicotine at 30 min if the content of polyalcohols is reduced four times in the current formulation recipe (black pentagram with error bars, Example 4).

FIG. 3 shows similar nicotine release even if the content of polyalcohols reduced to zero but with the addition of polysaccharides such as sodium alginate and moisturizing agents (open circle with error bars, Example 5).

FIG. 3 also shows batch-to-batch variation according to the present invention, Example 1. There is a relatively high batch-to-batch variation (filled circles, squares and diamonds with error bars) of the nicotine at 10 min.

A pouch based on nicotine polacrilex with reduced amount of buffer agents according to the present invention, Example 3, has the same release of nicotine at 30 min as compared with a pouch based on nicotine polacrilex according to the present invention, Example 1. It appears that a pouch based on nicotine polacrilex with reduced amount of buffer agents according to the present invention, Example 3, has similar release of nicotine at 30 min as compared with a reference pouch based on a nicotine salt such as nicotine tartrate. A pouch based on nicotine polacrilex with reduced amount of polyalcohols according to the present invention, Example 4, has slightly lower release of nicotine at 30 min as compared with a pouch based on nicotine polacrilex according to the present invention, Example 1. A pouch based on nicotine polacrilex with reduced amount of polyalcohols according to the present invention, Example 4, has higher nicotine release as compared with a reference pouch based on nicotine polacrilex.

The pH of the nicotine containing water phase from the test tube for the examined pouch samples of Example 1 was also measured. The obtained pH values are summarized in Table 4.

Table 4 shows that the pH for the pouch based on nicotine polacrilex according to the present invention is above pH 8.

The pH of the nicotine containing water phase from the test tube for the examined pouch samples of Example 1 and Example 5 was also measured. The obtained pH values are summarized in Table 4B.

Table 4B shows that the pH for the pouch based on nicotine polacrilex according to the present invention is above pH 8.

TABLE 4
pH values of the water phase containing nicotine in the test
tube for the examined pouch samples at specific timepoint.

| | |
|---|---|
| Specific timepoint, Min | pH values |

In-Vitro

| | |
|---|---|
| 0(non-used) | 8.62 |
| 10 | 8.47 |
| 20 | 8.42 |
| 30 | 8.49 |

TABLE 4B
pH values of the water phase containing nicotine in the test
tube for the examined pouch samples at specific timepoint.

| Specific timepoint, Min | pH values |
|---|---|
| In-Vitro | |
| | Examples |

| | 1 | 5 |
|---|---|---|
| 0(non-used) | 8.62 | 9.67 |
| 10 | 8.47 | — |
| 20 | 8.42 | — |
| 30 | 8.49 | 9.58 |

1.-16. (canceled)

17. An oral dosage pouch, comprising:
a powder composition, comprising:
nicotine;
at least one pH adjusting agent; and
one or more fillers, wherein at least one filler is microcrystalline cellulose in the amount of 40-80 wt % by weight of the powder composition;
wherein the powder composition has a solubility of less than 5 g/100 ml; and
a pouch which is permeable to saliva and to components of the powder composition once dissolved in saliva.

18. The pouch of claim 17, wherein the pouch is a non-woven pouch.

19. The pouch of claim 17, wherein the nicotine is in a nicotine source, and the nicotine source is 0.5-2 wt % by weight of the powder composition calculated as nicotine base.

20. The pouch of claim 17, wherein the nicotine is stabilized in the form of a nicotine salt.

21. The pouch of claim 20, wherein the nicotine salt is nicotine benzoate or nicotine maleate.

22. The pouch of claim 17, wherein the nicotine is bound to an ion exchange resin.

23. The pouch of claim 22, wherein the ion exchange resin is a weak cation exchange resin.

24. The pouch of claim 22, wherein the ion exchange resin is polacrilex.

25. The pouch of claim 17, wherein the pH adjusting agent is a buffer comprising carbonates, bicarbonates, borates, glycinates, ammonium, phosphates, hydroxides, tris, or mixtures thereof.

26. The pouch of claim 17, wherein the pH adjusting agent is 4-15 wt % by weight of the powder composition.

27. The pouch of claim 17, further comprising one or more polyalcohols, sweeteners, polysaccharides, cellulose, natural fibers, flavors, or mixtures thereof.

28. The pouch of claim 27, wherein at least one sweetener is present, the sweetener comprising one or more of mannitol, xylitol, maltitol, sucralose, acesulfam potassium, aspartame, steviol glycosides, or mixtures thereof.

29. The pouch of claim 17, wherein the solubility of less than 5 g/100 ml is a solubility in water at pH 7 and 25° C.

30. The pouch of claim 29, wherein the solubility of less than 5 g/100 ml is a solubility of less than 2.5 g/100 ml.

31. The pouch of claim 29, wherein the solubility of less than 5 g/100 ml is a solubility of less than 1.0 g/100 ml.

32. The pouch of claim 17, wherein the pouch has a nicotine release fraction of 50-100% after 30 min.

33. An oral dosage pouch, comprising:
a powder composition, comprising:
nicotine bound to a weak cation exchange resin;
at least one pH adjusting agent in the amount of 4-15 wt % by weight of the powder composition; and
one or more fillers, wherein at least one filler is microcrystalline cellulose in the amount of 40-80 wt % by weight of the powder composition;
wherein the powder composition has a solubility of less than 5 g/100 ml in water at pH 7 and 25° C.; and
a non-woven pouch containing the powder composition, wherein the pouch is permeable to saliva and to components of the powder composition once dissolved in saliva.

34. The pouch of claim 33, wherein the weak cation exchange resin is polacrilex.

35. The pouch of claim 33, wherein the pH adjusting agent is a buffer comprising carbonates, bicarbonates, borates, glycinates, ammonium, phosphates, hydroxides, tris, or mixtures thereof.

36. The pouch of claim 33, further comprising one or more polyalcohols, sweeteners, polysaccharides, cellulose, natural fibers, flavors, or mixtures thereof.
